(19) United States
(12) Patent Application Publication
(10) Pub. No.: US 2026/0053876 A1
(43) Pub. Date: Feb. 26, 2026

(54) COMPOSITIONS AND METHODS FOR PREVENTING, ALLEVIATING, OR TREATING LIVER DISEASE

(71) Applicants: EFIL BIOSCIENCE INC., Daejeon (KR); PURDUE RESEARCH FOUNDATION, West Lafayette, IN (US)

(72) Inventors: Kee-Hong KIM, West Lafayette, IN (US); Jeong-Hoon KIM, Daejeon (KR); Mu Lim CHOI, Daejeon (KR); Kongsik KIM, Daejeon (KR); Jaehyun Ju, West Lafayette, IN (US)

(21) Appl. No.: 19/252,440

(22) Filed: Jun. 27, 2025

Related U.S. Application Data
(60) Provisional application No. 63/664,852, filed on Jun. 27, 2024.

(51) Int. Cl. A61K 36/23; A61P 1/16

(52) U.S. Cl. CPC A61K 36/23; A61P 1/16; A61K 2236/13; A61K 2236/15; A61K 2236/17; A61K 2236/333; A61K 2236/37; A61K 2236/39

(57) ABSTRACT

The invention provides compositions containing aged parsnip extracts with enhanced polyphenol content and methods for their use in promoting liver health and treating metabolic liver diseases such as MASLD and MASH. The extracts are obtained via novel, mild heat-aging processes and exhibit anti-steatotic, anti-inflammatory, and antifibrotic effects in vivo.

### CROSS-REFERENCE TO RELATED APPLICATIONS

The present application claims priority to U.S. Application No. 63/664,852 filed on Jun. 27, 2024, which application is incorporated herein by reference.

### TECHNICAL FIELD

The present invention provides a composition comprising aged parsnip extracts and a method of using the composition to prevent, alleviate, or treat liver diseases.

### BACKGROUND

Mammals, including humans, suffer from various liver diseases caused by genetic and environmental factors. In recent decades, substantial progress has been made in developing drug therapies for liver diseases such as metabolic dysfunction-associated steatotic liver disease (MASLD) and metabolic dysfunction-associated steatohepatitis (MASH). Researchers have focused on discovering effective treatments that reduce hepatic lipid accumulation, suppress inflammatory responses, and prevent fibrosis.

As reported in, e.g., WO 2016/144862, U.S. Patent Application Publication No. 20140377222, U.S. Patent Application Publication No. 20140371271, U.S. Patent Application Publication No. 20140336113, U.S. Patent Application Publication No. 20140369965, U.S. Patent Application Publication No. 20200131129A1, U.S. Patent Application Publication No. US20220143003A1, U.S. Patent Application Publication No. 240066020A1, and U.S. Pat. No. 11,141,399B2, which are incorporated herein by reference, research on liver diseases is actively underway. Nevertheless, there remains a critical unmet need for innovative solutions to effectively prevent, alleviate, or treat these liver diseases.

### SUMMARY

The present invention provides a method for pre-treating raw parsnips into aged parsnips and extracting these aged parsnips to produce parsnip extracts having physiological activities higher than those of raw parsnips. Also, it provides a composition comprising the parsnip extracts having enhanced physiological activities. In addition, it provides a method for promoting liver health and/or preventing, improving, or treating liver by using the composition.

In one aspect, the present invention provides a composition for promoting liver health. More specifically, in one aspect, the present invention provides a composition for promoting liver health, the composition comprising an aged parsnip extract that is characterized in that the total content of at least one of the components of the aged parsnip extract is greater than that of unprocessed raw (natural) parsnips. For example, the component may be polyphenols. As a non-limiting example, the aged parsnip extract of the present invention may preferably have a total polyphenol content of at least 1.5 times, preferably at least 2 times, and more preferably at least 3 times more than the raw parsnips. The promotion of liver health may include preventing, improving, or treating diseases, disorders, conditions, or symptoms associated with liver diseases. Liver diseases may include MASLD and MASH.

In another aspect, the present invention provides a method for preparing the extract. More specifically, in another aspect, the present invention provides a method of preparing processed aged parsnip extracts having a total polyphenol content greater than (e.g., at least 2 times greater than) that of unprocessed raw (natural) parsnips. The preparation method includes the steps of heat drying raw parsnips for a predetermined period of time at a predetermined range of temperature, adding alcohol, water, or a mixture thereof to the thus heat-dried parsnips, filtering the supernatant after extraction, and removing unnecessary components. In accordance with an embodiment of the present invention, the heat drying step may be conducted to such an extent that the color of the raw parsnips changes to brown or black and that the total polyphenol content of the processed parsnips becomes greater than the total polyphenol content of the raw parsnips.

In accordance with an embodiment of the present invention, raw parsnips can be heat-dried at a predetermined range of temperature (or a predetermined temperature) for a predetermined period of time (or a predetermined time) to the point where the color of the raw parsnips changes to brown or black, and then the extraction process is applied to obtain the extract. As an example, the predetermined range of temperature can be adjusted to a temperature between about 50° C. and about 70° C., and the predetermined period can be adjusted to a period between about 10 and about 30 days. As another example, the predetermined range of temperature can be adjusted to a temperature between about 55° C. and about 65° C., and the predetermined c period can be adjusted to a period between about 10 and about 20 days. As another example, the predetermined range of temperature can be adjusted to a temperature between about 70° C. and about 90° C., and the predetermined period can be adjusted to a period between about 3 and about 10 days. As another example, the predetermined range of temperature can be adjusted to a temperature between about 85° C. and about 90° C., and the predetermined period can be adjusted to a period between 3 and 10 days. According to some other embodiments, the predetermined range of temperature and the predetermined period of time can be adjusted to such an extent that the color of the raw parsnips changes to brown or black and/or that the total polyphenol content of the processed parsnips becomes greater than the total polyphenol content of the raw parsnips.

The above and other aspects of the invention and representative embodiments of the invention are described in more detail below.

### BRIEF DESCRIPTION OF DRAWINGS

FIGS. 1A-1C illustrate the process of preparing aged parsnip (AP) extracts.

FIG. 2 outlines the animal study conducted according to an embodiment of the present invention.

FIG. 3 is a graph showing the effect of AP supplementation on body weight change in mice fed a high-fat, high-sucrose, and high-cholesterol (HF/HS/HC) diet for 17 weeks. “Cont” refers to the group fed a standard control diet (10% fat), and “HF/HS/HC+AP” refers to the group fed HF/HS/HC supplemented with 0.024% AP.

FIG. 4 is a graph showing the changes in fat and lean mass of mice in the Cont, HF/HS/HC, and HF/HS/HC+AP groups, assessed by Echo-MRI at the end of the study.

FIG. 5 is a graph showing the weight of individual fat pads (Epi: epididymal, Ing: inguinal, and Ret: retroperitoneal) in mice from the Cont, HF/HS/HC, and HF/HS/HC+AP groups, measured after euthanasia.

FIG. 6 is a graph showing the weight of individual heart, lung, and kidney tissues in mice from the Cont, HF/HS/HC, and HF/HS/HC+AP groups, measured after euthanasia.

FIG. 7 is a graph showing the weight of individual heart, lung, and kidney tissues in mice from the Cont, HF/HS/HC, and HF/HS/HC+AP groups, measured after euthanasia.

FIG. 8 is an image showing representative liver tissue images from mice in the Cont, HF/HS/HC, and HF/HS/HC+AP groups, measured after euthanasia. Scale bar: 1 cm.

FIG. 9 is an image showing the hematoxylin & eosin (H&E) staining results of the liver tissues from mice in the Cont, HF/HS/HC, and HF/HS/HC+AP groups, measured after euthanasia, according to an embodiment of the invention. Scale bar: 10 μm.

FIG. 10 is a graph showing the weight of the liver tissue isolated from mice in the Cont, HF/HS/HC, and HF/HS/HC+AP groups.

FIG. 11 is a graph showing mice's nonalcoholic fatty liver disease activity score (NAS) in the Cont, HF/HS/HC, and HF/HS/HC+AP groups.

FIG. 12 is a graph showing the alanine transaminase (ALT) activity in the plasma isolated from mice in the Cont, HF/HS/HC, and HF/HS/HC+AP groups.

FIG. 13 is a graph showing the aspartate aminotransferase (AST) activity in the plasma isolated from mice in the Cont, HF/HS/HC, and HF/HS/HC+AP groups.

FIG. 14 is a graph showing the triglyceride (TG) levels in the plasma isolated from mice in the Cont, HF/HS/HC, and HF/HS/HC+AP groups.

FIG. 15 an image showing the Sirius red staining results of the liver tissues from mice in the Cont, HF/HS/HC, and HF/HS/HC+AP groups, measured after euthanasia, according to an embodiment of the invention. Scale bar: 100 μm.

FIG. 16 a graph showing quantitative image analysis of liver fibrosis presented as the percent area (% area) and area (μm²) of Sirius red staining-positive liver fibrosis in the Cont, HF/HS/HC, and HF/HS/HC+AP group mice.

FIG. 17 a graph showing the thiobarbituric acid-reactive substances (TBARS) levels in the plasma isolated from mice in the Cont, HF/HS/HC, and HF/HS/HC+AP groups.

FIG. 18 a graph showing various pro-inflammatory cytokine (i.e., C5/C5a, eotaxin, ICAM-1, and IL-4) levels in the plasma isolated from mice in the Cont, HF/HS/HC, and HF/HS/HC+AP groups.

FIG. 19 is a graph representing the change of total polyphenol content of the parsnip extract of an embodiment of the present invention. AP 10D, AP 20D, and AP 30D refer to processed parsnip extracts obtained by pretreating (alternatively, aging or processing) for 10 days, 20 days, and 30 days, respectively, and Raw Parsnip refers to parsnip extract obtained without conducting such pretreatment.

### DETAILED DESCRIPTION

For the purpose of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “substantially” can allow for a degree of variability in a value or range, for example, within 90%, within 95%, or within 99% of a stated value or of a stated limit of a range.

In this document, the terms “a,” “an,” or “the” are used to include one or more than one unless the context clearly dictates otherwise.

The term “or” is used to refer to a nonexclusive “or” unless otherwise indicated.

In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

### I. Parsnip Extracts

In one aspect, the present invention provides parsnip extracts having physiological activities greater than raw parsnips. In accordance with an embodiment of the prevent invention, raw parsnips are pretreated to make aged or processed parsnips, and the aged or processed parsnips are subjected to an extraction process, thereby preparing the parsnip extracts of the invention. The parsnip extracts of the invention have physiological activities greater than raw parsnips.

A process of pretreating raw parsnips was reported in Plant Foods for Human Nutrition (2020) 75:292-297), but the process takes too much time (4 weeks) to complete and requires high temperature (80° C.) heat treatment and a high level of humidity (95%), which limits its application in industrial sites. There is an urgent need for a new method of pre-treating raw parsnips with a shorter period of time and milder process conditions.

According to embodiments of the present invention, it is possible to obtain parsnip extracts with significantly improved physiological activities compared to raw parsnips by pre-treating raw parsnips with mild process conditions and/or for a relatively short time.

In accordance with embodiments of the invention, raw parsnips are pretreated to make aged parsnips. The raw parsnips can be cut into pieces before pretreatment. The shape (e.g., round, spherical, cuboid, cube, etc.) and size (e.g., large, medium, small, etc.) can be adjusted accordingly as needed. During the pretreatment process, temperature, time, and/or pressure can be set appropriately.

For example, the temperature can suitably be heated to about 40˜90° C., 45˜85° C., 50˜80° C., 55˜75° C., or 60˜70° C. However, the above-listed ranges are only examples, and the temperature can be adjusted accordingly as needed. The heating can be suitably conducted for a predetermined period of time. For example, it can be heated for about 1˜30 days, 5˜25 days, 10˜20 days, or 13˜17 days. However, the above-listed ranges are only illustrative, and the heating period can be adjusted accordingly as needed. The pressure condition can suitably be adjusted appropriately during the heating. For example, it can be done under atmospheric pressure conditions or at high pressure. However, the above pressure conditions are only examples, and the pressure conditions can be adjusted accordingly as needed. For example, the heating and drying process can be conducted either wet or dry. If necessary, wet drying and dry drying can be used together.

After the pretreatment, water, alcohol, or a mixture thereof is added for extraction. The type of alcohol can be appropriately selected according to needs. Preferably, ethanol may be chosen. The concentration of alcohol can be adjusted as needed. Preferably, about 10˜45% alcohol can be used. Preferably, about 15˜40% alcohol can be used. Even more preferably, 20˜30% alcohol can be used. However, these alcohol concentration ranges are only illustrative and can be adjusted as needed. Preferably, water, alcohol, or a water/alcohol mixture can be used in greater amount than the raw parsnips (e.g., 2, 3, 4, 5, 6, . . . times raw parsnips).

Thereafter, the aged parsnips are extracted in a stirring shaker. The temperature and time for the extraction can be selected according to needs. For example, they can be extracted at room temperature for 24 hours. Supernatants are filtered after the extraction, and additional extraction can be conducted, if needed. All supernatants obtained in the extraction process are collected, and the collected supernatants are concentrated under reduced pressure to remove residual organic solvents and treated with nitrogen to remove residual moisture.

### II. Compositions

In another aspect, the present invention provides a composition comprising the parsnip extracts having physiological activities greater than raw parsnips for use in preventing, improving (alleviating), or treating liver diseases, disorders, conditions, or symptoms.

As used herein, the term “composition” is intended to contain the extract obtained by the above-described methods or contain any other product resulting directly or indirectly from the extract. The term “composition” is intended to contain a combination of at least one of the extract and at least one of the product resulting from the extract, a combination of two or more of the extracts, or a combination of two or more of the products resulting from the extracts.

As used herein, the term “composition” refers to a mixture of a pharmaceutically or therapeutically active component (ingredient) with one or more other components, which may be chemically or biologically active or inactive. Such components may include, but not limited to, carriers, stabilizers, diluents, dispersing agents, suspending agents, thickening agents, excipients, and adjuvants.

Any suitable pharmaceutically acceptable carriers, stabilizers, diluents, dispersing agents, suspending agents, thickening agents, excipients, and adjuvants known to those of ordinary skill in the art for use in pharmaceutical compositions may be selected and employed in the compositions described herein. As used herein, the term “acceptable” with respect to a formulation, composition, or ingredient, as used herein, means having no persistent detrimental effect on the general health of the subject being treated. As used herein, the term “carrier” refers to chemical or biological material that can facilitate the incorporation of a therapeutically active ingredient(s) into cells or tissues. Examples of the pharmaceutically acceptable carrier include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, mineral oil, and the like, but it is not limited thereto.

Suitable excipients may include, for example, water, pharmaceutically acceptable organic solvents such as paraffins (e.g., petroleum fractions), vegetable oils (e.g. groundnut or sesame oil), mono- or polyfunctional alcohols (e.g., ethanol or glycerol), carriers such as natural mineral powders (e.g., kaoline, clays, talc, chalk), synthetic mineral powders (e.g., highly dispersed silicic acid and silicates), sugars (e.g., cane sugar, lactose and glucose), emulsifiers (e.g., lignin, spent sulphite liquors, methylcellulose, starch and polyvinylpyrrolidone), and lubricants (e.g., magnesium stearate, talc, stearic acid and sodium lauryl sulphate).

The compositions described herein may be in the form of a solid, liquid, or gas (aerosol). For example, they may be in the form of tablets (coated tablets) made of, for example, collidone or shellac, gum Arabic, talc, titanium dioxide or sugar, capsules (gelatin), solutions (aqueous or aqueous-ethanolic solution), syrups containing the active substances, emulsions or inhalable powders (of various saccharides such as lactose or glucose, salts and mixture of these excipients with one another), and aerosols (propellant-containing or -free inhale solutions). Also, the compositions described herein may be formulated for sustained or slow release.

### III. Methods

In still another aspect, the present invention provides a method of using a composition comprising the parsnip extracts having enhanced physiological activities to promote liver health and/or to prevent, improve, or treat liver symptoms, conditions, or diseases.

As used herein, the term “treat,” “treating” or “treatment” refers to methods of alleviating, abating, ameliorating, inhibiting, relieving, stopping, or preventing a disease, disorder, condition, or symptom. The term also refers to methods of alleviating, abating, ameliorating, inhibiting, relieving, stopping, or preventing the underlying causes of the disease, disorder, condition, or symptom.

As used herein, the term “subject” or “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fishes and the like.

As used herein, the term “administration” or “administering” of the subject composition refers to providing a composition of the invention and/or a prodrug thereof to a subject in need of treatment.

As used herein, the term “effective amount” or “therapeutically effective amount” refer to a sufficient amount of an active ingredient(s) described herein being administered which will, for example, relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition as disclosed herein required to provide a clinically significant decrease in disease symptoms. An appropriate “effective” amount in any individual case may be determined using techniques, such as a dose-escalation study.

In addition, such compositions may be administered singly or in combination with one or more additional functional or therapeutic agents. The methods of administration of such compositions may include, but are not limited to, intravenous administration, inhalation, oral administration, rectal administration, parenteral, intravitreal administration, subcutaneous administration, intramuscular administration, intranasal administration, dermal administration, topical administration, ophthalmic administration, buccal administration, tracheal administration, bronchial administration, sublingual administration or optic administration. The method of administration of the composition is determined according to the degree of symptoms. In addition, the dosage of the active ingredient in the composition may vary depending on the route of administration, the severity of the disease, the age, the sex, and weight of the patient, and may be administered once to several times a day.

Compositions provided herein may be administered by way of known formulations, including tablets, capsules or elixirs for oral administration, suppositories for rectal administration, sterile solutions or suspensions for parenteral or intramuscular administration, lotions, gels, ointments or creams for topical administration, and the like. In some embodiments, such compositions are formulated as tablets, pills, capsules, a liquid, an inhalant, a nasal spray solution, a suppository, a solution, a gel, an emulsion, an ointment, eye drops, or ear drops.

The therapeutically effective amount may vary depending on, among others, the disease indicated, the severity of the disease, the age and relative health of the subject, the potency of the compound administered, the mode of administration and the treatment desired. The required dosage will also vary depending on the mode of administration, the particular condition to be treated and the effect desired.

The above and other embodiments and uses will be apparent to one skilled in the art in light of the present disclosures. The following examples are provided merely as illustrative of various embodiments and shall not be construed to limit the invention in any way.

### EXAMPLES

Hereinafter, the present invention will be described in more detail through examples. These examples are only intended to illustrate the present invention in more detail, and it will be apparent to those skilled in the art that the scope of the present invention is not limited by these examples according to the gist of the present invention.

### Example 1: Preparation of Post-Harvest Aged Parsnip Extract

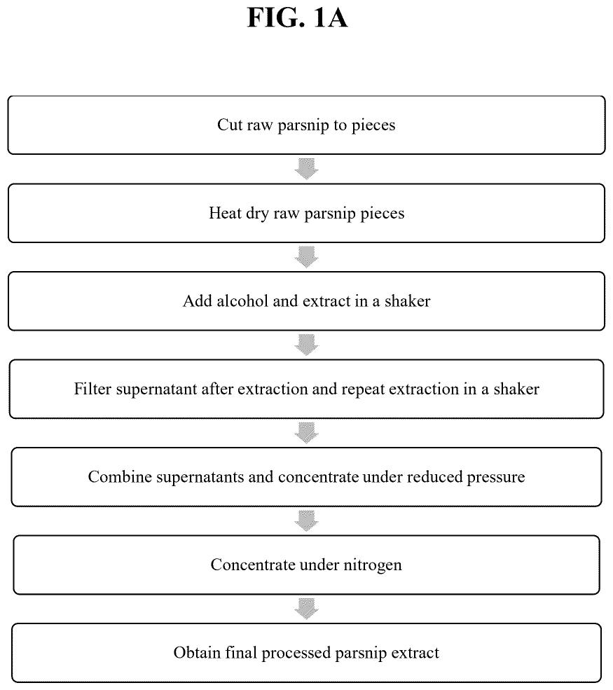

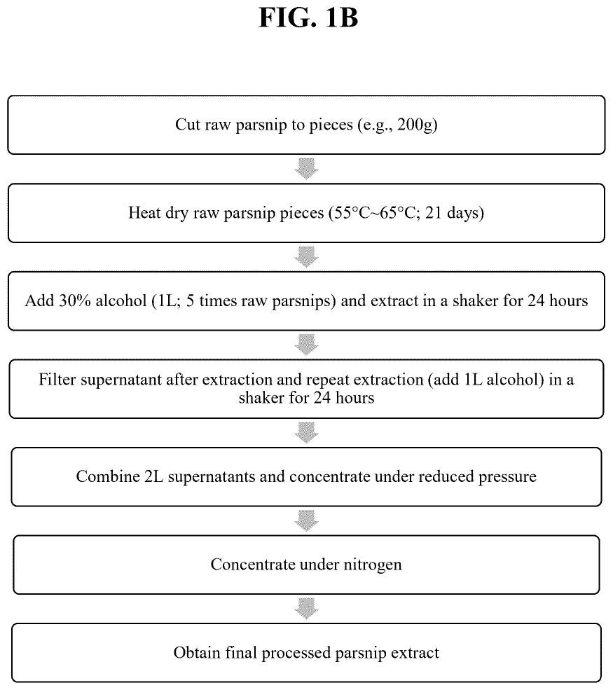

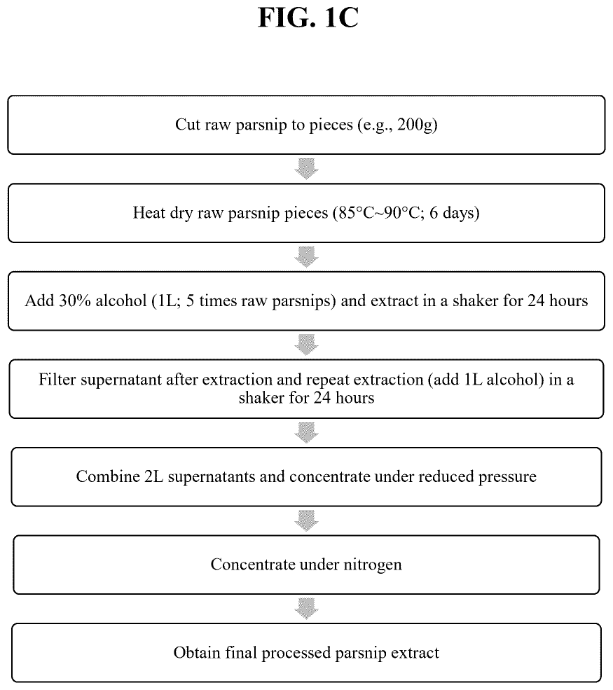

FIG. 1A illustrates the process of preparing post-harvest aged parsnip extract of the present invention. In an embodiment, as illustrated in FIG. 1B, raw parsnips were peeled, washed, and cut to pieces so that each piece was about 200 g. The parsnip pieces were placed in a drying chamber maintained at 55° C.˜65° C. and heat-dried for at least 10 days. Alternatively, the parsnip pieces were placed in a drying chamber maintained at 85° C.˜90° C. and heat-dried for 6 days. 30% alcohol (or a mixture of alcohol and water) was added to the heat-dried parsnip pieces. The extraction was performed at room temperature (20° C.˜25° C.) for 24 hours in a stirred incubator (125 rpm). The supernatant was filtered by filter paper (Whatman No 4). The parsnips remaining on the filter paper were extracted one more time under the same conditions, and the supernatant was collected one more time. The thus-obtained final processed aged parsnip extract was subjected to reduced pressure extraction to remove residual organic solvents and then subjected to nitrogen treatment to remove residual moisture.

In another embodiment, as illustrated in FIG. 1C, raw parsnips were peeled, washed, and cut to pieces so that each piece was about 200 g. The parsnip pieces were placed in a drying chamber maintained at 85˜90° C. and heat-dried for 6 days. 30% alcohol (or a mixture of alcohol and water) was added to the heat-dried parsnip pieces. The extraction was performed at room temperature (20˜25° C.) for 24 hours in a stirred incubator (125 rpm). The supernatant was filtered by a filter paper (Whatman No 4). The parsnips remaining on the filter paper were extracted one more time under the same conditions, and the supernatant was collected one more time. The thus-obtained final processed parsnip extract was subjected to reduced pressure extraction to remove residual organic solvents and then subjected to nitrogen treatment to remove residual moisture.

### Example 2: Determination of Total Polyphenol Content

2.5 mg/mL, 5.0 mg/mL, and 10.0 mg/mL of the final processed parsnip extracts prepared in Example 1 were prepared. 100 μL of 2.5 mg/mL of the final processed parsnip extract and 200 μL of 2% Na2CO3 were reacted for 3 minutes, 100 μL of 5.0 mg/mL of the final processed parsnip extract and 200 μL of 2% Na2CO3 were reacted for 3 minutes, and 100 μL of 10.0 mg/mL of the final processed parsnip extract and 200 μL of 2% Na2CO3 were reacted for 3 minutes. To the resulting mixtures, 10 μL of 50% Folin-ciocalteu reagent was added and reacted for 3 minutes, and then absorbance was measured at 720 nm. As for a blank, the organic solvent (i.e., 15% DMSO), which was used to dissolve parsnip extract, was mixed and reacted with 2% Na2CO3 for 3 minutes. For the control, 50% Folin-ciocalteu reagent was added to the blank (i.e., the mixture of 15% DMSO and 2% Na2CO3) and reacted for 3 minutes. The content of total polyphenols was shown against a calibration curve using gallic acid.

As shown in FIG. 19, processed parsnips had a total polyphenol content that was approximately twice as high as raw parsnips as early as day 10, and under the 20-day and 30-day processing conditions, the total polyphenol content was equivalent to or higher than that of red ginseng, depending on the concentration. There was no significant difference between processed parsnips manufactured under 20-day and 30-day processing conditions. Taken together, the total polyphenol content of processed parsnips prepared by the method of the present invention was significantly higher than the total polyphenol content of raw parsnips (FIG. 19). For example, the total polyphenol content of the processed parsnip obtained by processing 20 or longer days was similar to or greater than that of commercially available red ginseng. Processed parsnips with such an elevated level of total polyphenol content could be obtained in a fairly short time.

### Example 3: Animal Study

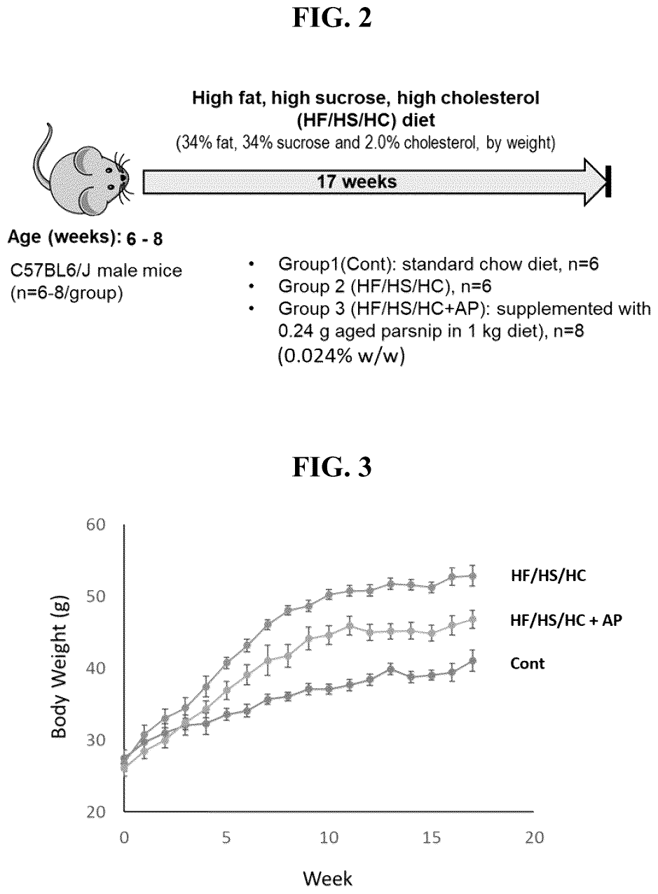

Six to eight-week-old male C57BL/6J mice were obtained from the Jackson Laboratory (Bar Harbor, ME, USA). Following a week of acclimatization, mice were maintained under a 12-hour light/dark cycle and had free access to food and water for 17 weeks of the experimental period. As illustrated in FIG. 2, mice were fed a standard control diet (10% fat), a high-fat, high-sucrose, and high-cholesterol (HF/HS/HC) diet (34% fat, 34% sucrose, 2.0% cholesterol), and an HF/HS/HC diet containing 0.024% (w/w) aged parsnip extract (AP) (HF/HS/HC+AP) for 17 weeks. HFSCD contains sucrose (350.0 g/kg), casein (200.0 g/kg), L-cystine (3.0 g/kg), lard (305 g/kg), soybean oil (40.0 g/kg), insoluble fiber (50.0 g/kg), AlN-93 mineral mix (40.0 g/kg), AlN-93 vitamin mix (10.0 g/kg), t-butylhydroquinone (0.008 g/kg) and choline bitartrate (2.0 g/kg). At the end of the feeding period, mice were starved for 8 hr and anesthetized carbon dioxide. Blood was collected in a tube containing 3.5 mg EDTA from the mice by cardiac puncture for serum preparation. Livers were snap-frozen in liquid nitrogen and stored at −80° C. until used to study gene expression or stored in 10% formalin for histological analysis.

Blood biochemistry: Plasma lipids were determined enzymatically using a Cholesterol Reagent and triglyceride (TG) assay kit for total cholesterol (TC) and TG, respectively. Plasma alanine transaminase (ALT) activity and aspartate aminotransferase (AST) activity were measured using assay kits. A Liquid Glucose (Oxidase) reagent set was used to determine plasma glucose concentrations according to the manufacturer's protocol. Liver TC and TG were measured enzymatically from lipids extracted by Folch's methods. Hepatic thiobarbituric acid-reactive substances (TBARS), considered oxidative stress markers, were measured using an assay kit (Cayman Chemical). The protein content of various pro-inflammatory cytokines in the plasma samples obtained from the test animals was analyzed using the Proteome Profiler Moues Cytokine Array kit (R&D Systems).

Histology of liver: Mouse liver sections were fixed in 4% paraformaldehyde and embedded in paraffin. Sections 4 μm thick were stained with hematoxylin and eosin (H&E), and Sirius red staining according to standard methods. Images were acquired using digital slide scanning (Aperio Technologies), and ImageJ (National Institutes of Health) analyzed the stained area percentage. The nonalcoholic fatty liver disease (NAFLD) activity score (NAS) was calculated as the unweighted sum of the scores for macrovesicular steatosis (0-3), microvesicular steatosis (0-3), and inflammation (0-3). A microscopic examination was performed by a board-certified veterinary pathologist, and the interpretation was based on standard histopathological morphology, which NAS adapted for rodents.

Statistical analysis: All data are presented as mean±SEM. Statistical analyses were performed with GraphPad Prism 5 program (GraphPad Software, Inc.). Two-tailed Student t-tests or ANOVA followed by the Tuckey post hoc test were performed to analyze the statistical significance of the results. P<0.05 was considered statistically significant.

### Example 4: Body Weight, Body Fat Weight, and Liver Weight

The body weights of the mice in the HF/HS/HC diet were significantly higher than those of the control (Cont) group. The mice fed with an HF/HS/HC diet supplemented with 0.024% AP (HF/HS/HC+AP) displayed significantly decreased body weight, starting from 5 weeks on the experimental diets (FIG. 3).

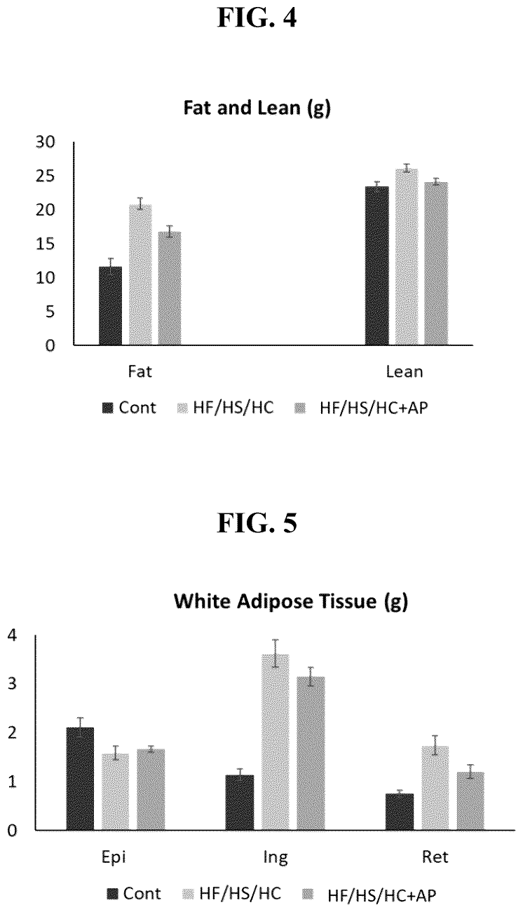

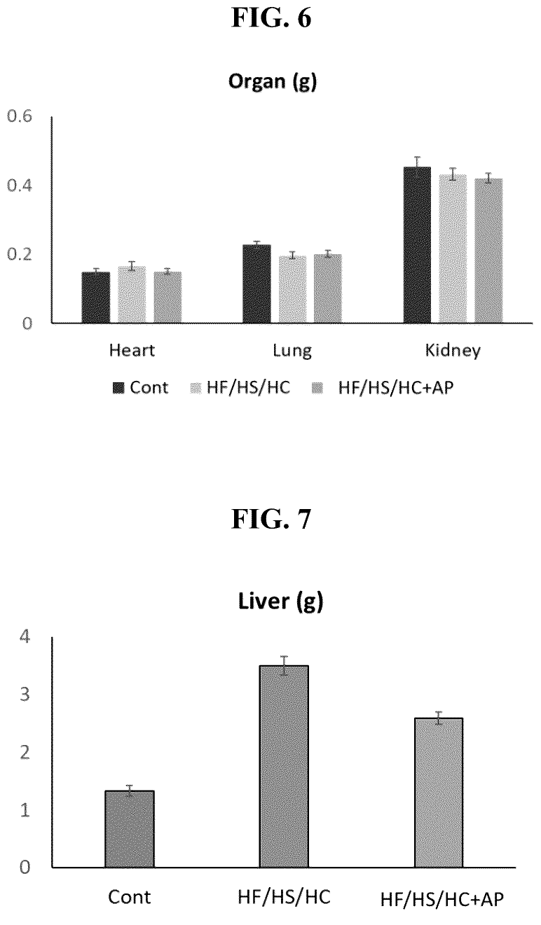

After 17 weeks on experimental diets, the HF/HS/HC+AP group showed 14% decrease in body weight compared to the HF/HS/HC group. Mice in the HF/HS/HC+AP group displayed 21% less fat mass and 8.8% less lean mass than those in the HF/HS/HC group as measured by Echo-MRI (FIG. 4). While there were significant decreases in retroperitoneal (Ret) fat pad weight (FIG. 5) in the HF/HS/HC+AP group when compared to the HF/HS/HC group, no significant differences in heart, lung and kidney weight were observed between the two groups (FIG. 6).

Aged parsnip extract reduced body weight, body fat weight, and liver weight gain in a mouse model of diet-induced metabolic dysfunction-associated steatotic liver disease (MASLD) and metabolic dysfunction-associated steatohepatitis (MASH).

### Example 5: MASLD

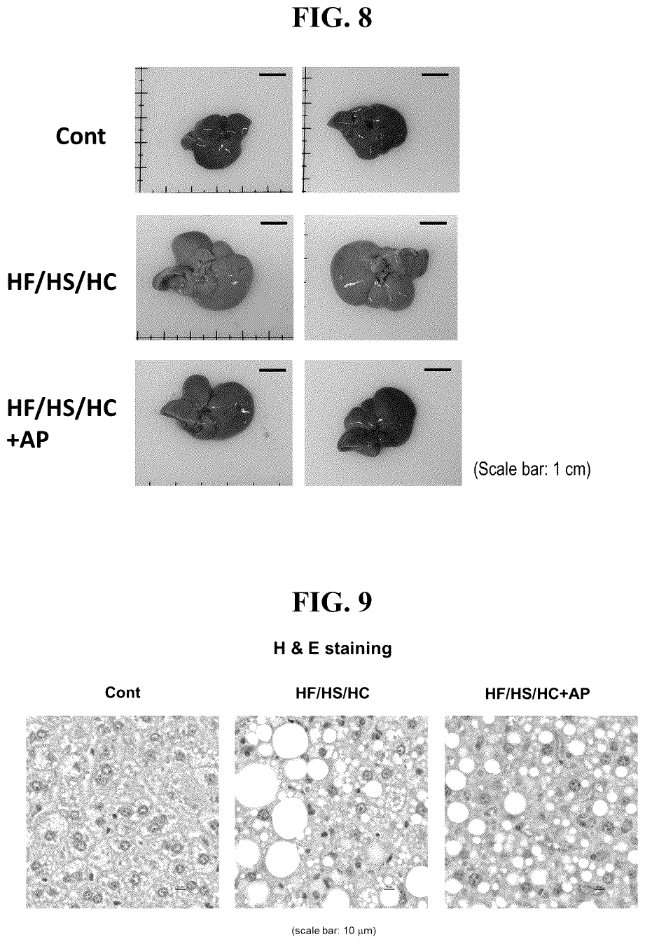

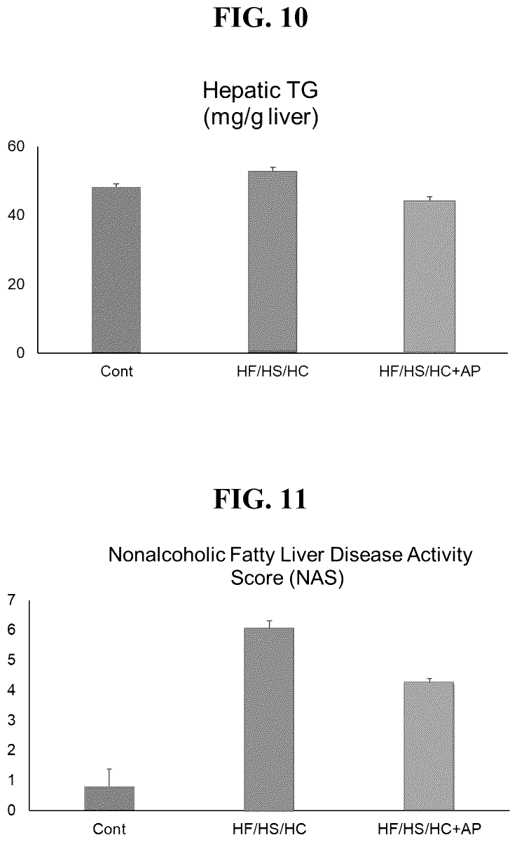

Next, we evaluated the effect of aged parsnip supplementation on MASLD. Liver weight was significantly higher in the HF/HS/HC group than in the Cont group. However, the liver weight was 26% lower in the HF/HS/HC+AP group than in the HF/HS/HC group (FIG. 7). Mice livers in the HF/HS/HC group were larger in size and had a lighter color, compared to the relative healthy red color of the mice in the HF/HS/HC group (FIG. 8). Evidently, H&E staining of the liver tissue showed that there was markedly higher lipid accumulation in the liver of HF/HS/HC mice compared to the Cont group, while the HF/HS/HC+AP mice showed lower lipid accumulation than HF/HS/HC mice (FIG. 9). Consistently, AP supplementation attenuated the liver TG levels in the HF/HS/HC+AP group compared to the HF/HS/HC group (FIG. 10). Nonalcoholic fatty liver disease activity score (NAS) was calculated for each liver tissue as the sum of the scores of macrovesicular steatosis, microvesicular steatosis and hepatic inflammation. As shown in FIG. 11, the livers from the HF/HS/HC group revealed a higher NAS (NAS=6) score with increased hepatic macrovesicular steatosis, microvesicular steatosis and inflammation. However, the HF/HS/HC+AP group showed reduced NAS (NAS=4.2).

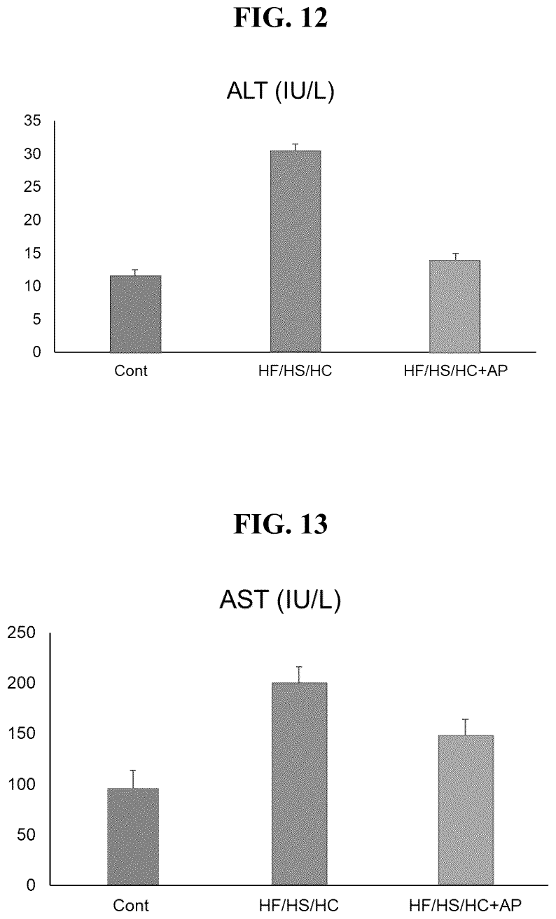

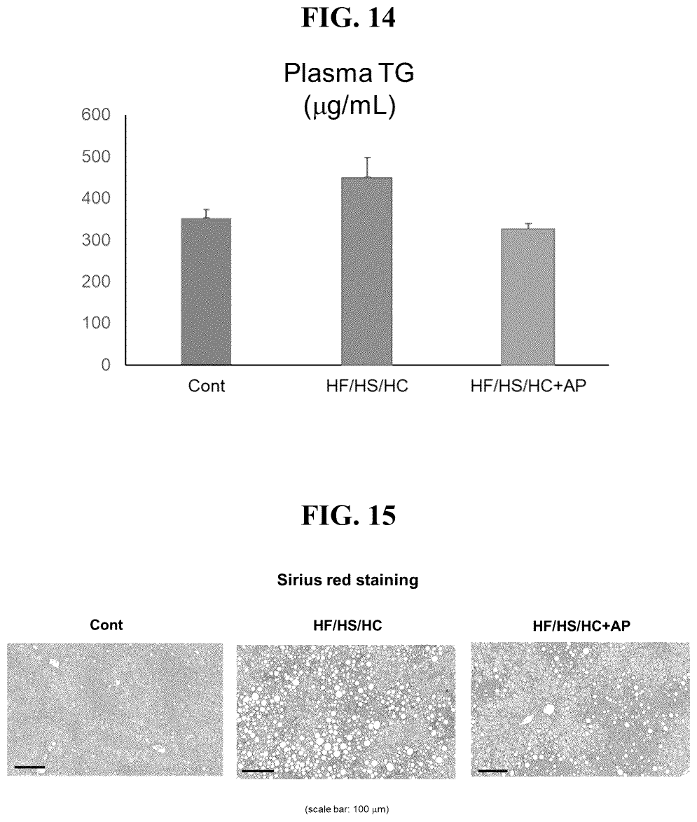

Plasma levels of ALT and AST, markers of liver injury, were significantly higher in the HF/HS/HC group compared to the Cont. However, AP supplementation in the HF/HS/HC+AP group markedly decreased plasma ALT (FIG. 12) and AST (FIG. 13) levels compared to those in the HF/HS/HC group. Moreover, AP supplementation in the HF/HS/HC+AP group markedly decreased plasma TG levels compared to those in the HF/HS/HC group (FIG. 14).

### Example 6: MASH

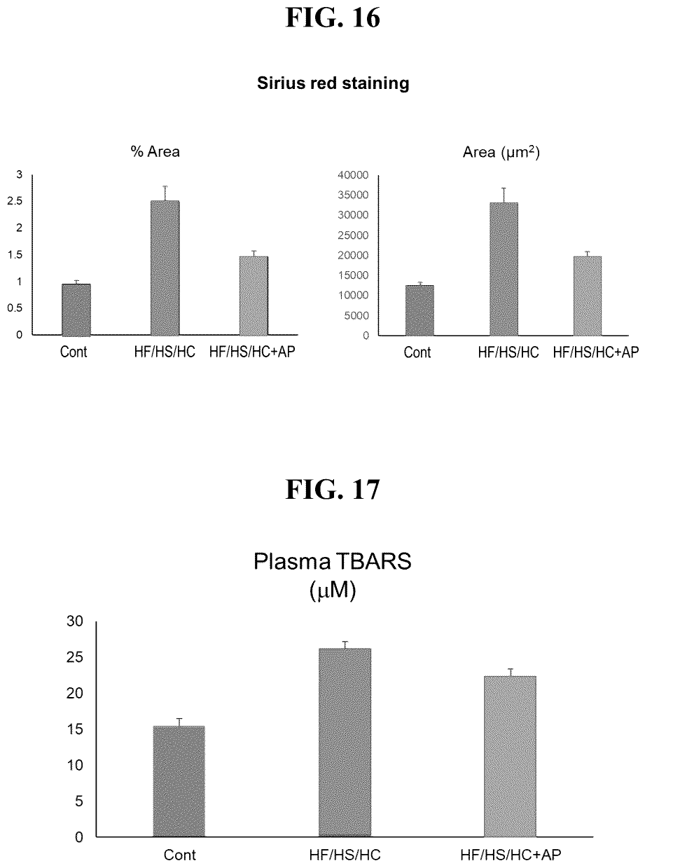

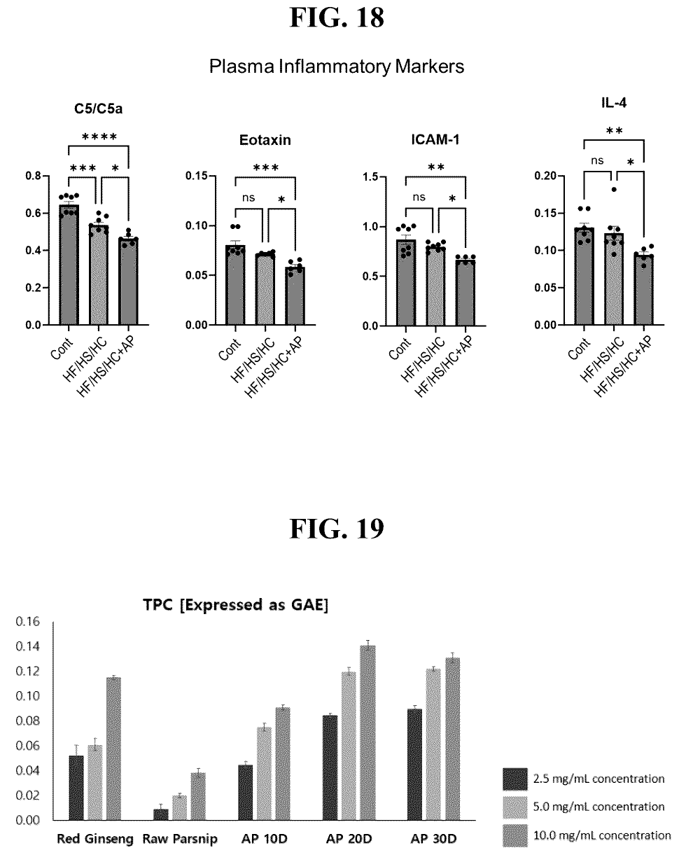

MASLD can progress to MASH, characterized by fibrosis and inflammation in the liver. The HF/HS/HC mice showed markedly increased fibrosis in the liver compared with the Cont mice as judged by Sirius Red staining images (FIG. 15) and quantitative calculation of Sirius Red positive fibers (FIG. 16). However, AP supplementation in the HF/HS/HC+AP group markedly decreased Sirius Red positive fibers (FIGS. 14 and 15). Liver oxidative stress is a key factor in the development of MASH. Thus, we determined the levels of liver oxidative stress in these mice by measuring them with TBARS. Evidently, mice in the HF/HS/HC group exhibited elevated levels of TBARS (FIG. 17). However, AP supplementation resulted in a decrease in the TBARS level. Progression of MASH is accompanied by an increase in inflammation. We found that HF/HS/HC diet-induced inflammatory cytokines in the plasma (i.e., C5/C1, eotaxin, ICAM-1 and IL-4) were blunted by AP supplementation (FIG. 18).

1. A composition comprising an aged parsnip extract that has a total polyphenol content greater than that of unprocessed raw parsnips, for preventing, improving, or treating disorders, conditions, or symptoms associated with liver disease.

2. The composition of claim 1, wherein the liver disease is fatty liver disease.

3. The composition of claim 1, wherein the liver disease is metabolic dysfunction-associated steatotic liver disease (MASLD).

4. The composition of claim 1, wherein the liver disease is metabolic dysfunction-associated steatohepatitis (MASH).

5. The composition of claim 1, wherein the aged parsnip extract has a total polyphenol content that is at least twice that of the unprocessed raw parsnips.

6. The composition of claim 1, further comprising one or more additional functional or therapeutic agents.

7. The composition of claim 1, wherein the composition is administered singly or in combination with one or more additional functional or therapeutic agents.

8. The composition of claim 1, wherein the composition is administered via a route selected from the group consisting of intravenous, inhalation, oral, rectal, parenteral, intravitreal, subcutaneous, intramuscular, intranasal, dermal, topical, ophthalmic, buccal, tracheal, bronchial, sublingual, and optic administration.

9. The composition of claim 1, wherein the composition is formulated for administration in a form selected from the group consisting of a tablet, pill, capsule, liquid, inhalant, nasal spray solution, suppository, sterile solution, suspension, gel, emulsion, ointment, lotion, cream, eye drop, and ear drop.

10. The composition of claim 1, wherein the aged parsnip extract is prepared by pretreating raw parsnips for a predetermined period of time at a predetermined range of temperature to the extent that the color of the raw parsnips changes to brown or black to make aged parsnips and extracting the aged parsnips.

11. The composition of claim 5, wherein the predetermined temperature is in the range of 50˜65° C. and the predetermined time is in the range of 10˜20 days.

12. The composition of claim 5, wherein the predetermined temperature is in the range of 85˜90° C. and the predetermined time is in the range of 3˜10 days.
